(19) United States
(12) Patent Application Publication
(10) Pub. No.: US 2007/0077258 A1
(43) Pub. Date: Apr. 5, 2007

(54) ADMINISTRATION OF GLUTATHIONE (REDUCED) VIA INTRAVENOUS OR ENCAPSULATED IN LIPOSOME FOR THE AMELIORATION OF TNF-alpha EFFECTS AND FLU-LIKE VIRAL SYMPTOMS AND TREATMENT AND PREVENTION OF VIRUS

(76) Inventor: F. Timothy Guilford, Palo Alto, CA (US)

Correspondence Address: BROOKE SCHUMM III, ONE NORTH CHARLES STREET, SUITE 2450, BALTIMORE, MD, 21201, US

(21) Appl. No.: 11/277,845

(22) Filed: Mar. 29, 2006

Related U.S. Application Data
(60) Provisional application No. 60/594,324, filed on Mar. 29, 2005.

(51) Int. Cl. A61K 39/145; A61K 38/05; A61K 9/127; A61K 31/13; A61K 33/04

(52) U.S. Cl. 424/209.1; 424/702; 424/450; 514/18; 514/662

(57) ABSTRACT

The invention is a method of treatment of the symptoms related to inflammation that accompanies the release of Tumor Necrosis Factor-alpha in diseases such as viral infection such as those affecting the respiratory tract by providing systemic glutathione (reduced) by oral administration of glutathione (reduced) in a liposome encapsulation or by the intravenous administration of reduced glutathione. The administration of a therapeutically effective amount of oral liposomal glutathione (reduced) results in improvement of symptoms of disease induced by the release of TNF-α in infectious disease states such as respiratory and other viruses. The product is novel in that it is stable across the temperature ranges encountered in shipping and does not need to be refrigerated for storage. Compounds enhancing the effect of the liposomal glutathione as well as intravenous glutathione are contemplated such as Selenium.

### CONTINUATION DATA

This application relies on the priority of U.S. Provisional 60/594,324 of the same name as this invention filed Mar. 29, 2005, and is a continuation-in-part for all countries required, including the United States of America.

### SUMMARY OF INVENTION

The invention is the use of a therapeutically effective amount of glutathione (reduced) in a liposome encapsulation capable of administration in an oral form or intravenously while effectively enhancing the cellular glutathione pathway, to improve symptoms of viruses, and associated diseases, particularly those diseases characterized by excess TNF-α and for the treatment and prevention of virus, particularly influenza. Further, the invention is stable for extended periods at room temperature, that is, without refrigeration.

### TECHNICAL FIELD

The invention relates to the field of delivery of a nutrient substance, glutathione in the biochemically-reduced form, used in a sufficient amount to improve the symptoms related to virus infection. The delivery may also be accomplished either intravenously or in a liposome encapsulation via absorption across the mucosa of the nose, mouth, gastrointestinal tract, or after topical application for transdermal, or intravenous infusion.

### BACKGROUND

The tripeptide L-glutathione (GSH) (gamma-glutamyl-cysteinyl-glycine) is well known in biological and medical studies to serve several essential functions in the cells of higher organisms such as mammals. It is functional when it appears in the biochemical form known as the reduced state (GSH). When oxidized, it forms into a form known as a dimer (GSSG).

Glutathione in the reduced state (GSH) functions as an antioxidant, protecting cells against free-radical mediated damage, a detoxifying agent by transporting toxins out of cells and out of the liver, and as a cell signal, particularly in the immune system.

The use of the term “glutathione” or “glutathione (reduced)” will refer to glutathione in the reduced state.

Influenza, also known as the flu, is a contagious respiratory illness caused by influenza viruses. It can cause mild to severe illness, and at times can lead to death. While most healthy people recover from the flu without complications, some people, such as older people, young children, and people with certain health conditions, are at high risk for serious complications from the flu. http://www.cdc.gov/flu/keyfacts.htm

Episodes of influenza tend to occur during the winter months with large segments of the population becoming rapidly affected. Severe epidemics related to influenza have occurred. Supportive therapy has been the traditional treatment of influenza symptoms. More recently, the use of pharmacologic agents has been developed. To date, there has been no approach which uses nutrient supplementation to support the immune mechanisms involved in the response to virus and the subsequent development of symptoms related to the body fighting virus infection in general and influenza in particular. Further, there is no approach which causes a shift in immune response from a Th-2 toward a Th-1 response, which would have the consequent effect of ameliorating the symptoms while in fact stimulating the immune system to more effectively combat the virus.

The influenza virus is a large RNA virus. The virus contains an unusual genomic structure of RNA segments. These segments reshuffle upon each cycle of infection, which has made it difficult to create a single reusable vaccine.

As the antigenic structure of the virus changes with each yearly cycle of infection, the flu vaccine must be prepared yearly from information received from around the world as to what the prevalent antigens may be. Thus, flu vaccination is not completely effective, is costly and has associated risks. A news wire service report on from Feb. 18, 2005 reported a CDC study that showed the influenza vaccine for the 2004-5 season failed to provide protection against the Fujian flu strain that caused the most cases of the flu in the 2005 flu season. One analysis of the data suggested that the vaccine protected only 1 to 14 percent of the participants in the study (http://www.sptimes.com/2004/01/15/Worldandnation/Study_Latest_flu_vac.shtml). The invention presents methods that can increase the individual's defense against the symptoms of influenza that can work with or without the flu vaccine.

Commonly, the flu includes the sudden onset of symptoms such as:

Fever (usually high)

Headache

Tiredness (can be extreme)

Cough

Runny or stuffy nose

Body aches

Diarrhea and vomiting also can occur but are more common in children.

Sore throat

These symptoms are referred to as “flu-like symptoms.” A number of different illnesses, including the common cold, can have similar symptoms.

At present there is no rapid mechanism for determining the etiology of symptoms included in the list above. While, when these occur they are lumped into the general category of being flu-like, there is no rapidly available test to specifically identify the etiology of the symptoms called influenza. Thus, the treatment choice depends on the clinical presentation of the symptoms The symptoms described above can be associated with many viruses such as picorna virus, or in particular, rhinovirus (Medical Microbiology 4th Edition, Ed. Sam Brown, University of Texas Medical Branch). Additionally, it is known that many cases of viral hepatitis are not diagnosed because the symptoms are vague and similar to a flu-like illness. Thus, flu-like illness symptoms are common to many viral diseases, including the virus related to the illness associated with the influenza virus. Thus the term flu-like or influenza will be used to describe the general group of symptoms related to viruses. The common stimulus to the symptom picture is found in the similarity of immune response to viral infections. This invention is meant to modulate the common symptoms manifested from the body's response to the infectious agents or diseases referenced in this paragraph.

It is commonly agreed that the current most effective prevention of influenza is the yearly flu vaccine. In addition there are medications such as amantadine, rimantadine, and oseltamivir (“TAMIFLU”® (Registered ™ of Roche)), which are each approved for use in preventing or early treatment of the flu, and are often administered based on the symptom presentation of a flu-like illness. While these medications are useful for the flu, they are effective against other viruses as well. They are most effective if given before exposure or at the earliest onset of the symptoms. The medications have a variety of undesirable side effects. Their use may be reviewed at http://www.nlm.nih.gov/medlineplus/druginfo/uspdi/202024.html

Jones et al, U.S. Pat No. 6,013,632, Jan. 11, 2000, have described the use of glutathione for the prevention or treatment of in-vitro cell lines infected with the influenza virus. The prevention of replication of flu virus was demonstrated in Madin-Darby Canine Kidney (MDCK) Cells, and Normal Human Small Airway Epithelial Cells (SAEC). The protection afforded to cell lines was observed even without the introduction of glutathione into the cells.

While Jones et al, U.S. Pat. No. 6,013,632 claim the use of glutathione in drink or lozenge to treat influenza virus, there is no demonstration of efficacy in a human nor is there a claim for the alleviation of flu-like symptoms. Glutathione is poorly absorbed or destroyed when administered orally with aspirations for gastrointestinal absorption, and appears to not be absorbed at all through mucous membranes, or transdermally, or upon oral administration.

Mark et al, U.S. Pat. No. 5,214,062, May 25, 1993, claim the use of a combination including an intracellular glutathione stimulator chosen from the group consisting of: L-2-oxothiazolidine-4-carboxylate, glutathione, and glutathione esters; and an omega-3 fatty acid source comprising at least 1.5% of the total calories for treating immune disorders, inflammation, and or chronic infections. There is no claim for the use of glutathione alone for the stated purposes or the modulation of symptoms of the viruses such as influenza.

The present invention claims the use of glutathione (reduced) administered either in an intravenous solution or in a liposomal preparation of glutathione, (reduced) for the treatment of virus related symptoms such as influenza symptoms and infection in both the chronic state and early, acute onset state of the illness.

A liposome is a microscopic fluid-filled pouch whose walls are made of one or more layers of phospholipid materials identical to the phospholipid that makes up cell membranes. Lipids can be used to deliver materials such as drugs to the body because of the enhanced absorption of the liposome. The outer wall of the liposome is fat soluble, while the inside is water-soluble. This combination allows the liposome to become an excellent method for delivery of water-soluble materials that would otherwise not be absorbed into the body. A common material used in the formation of liposomes is phosphatidylcholine, the material found in lecithin. A more detailed description of the constituents of this invention is provided.

Replacing glutathione in human deficient states has been difficult because of the lack of direct absorption of glutathione after oral administration. Glutathione is a water-soluble peptide. This characteristic of glutathione is thought to prevent its absorption into the system after oral ingestion of glutathione. The fate of direct oral ingestion of glutathione has been demonstrated in a clinical study showing that 3 grams of glutathione delivered by oral ingestion does not elevate plasma glutathione levels.

The inventor Guilford filed a provisional application Ser. No. 60/522,785 on Nov. 7, 2004 entitled “Liposomal Formulation for Oral Administration of Glutathione (Reduced)” which is adopted and incorporated herein by reference. Presently pending is Guilford, the inventor herein, U.S. Utility application Ser. No. 10/289,934 filed Nov. 7, 2002 entitled Systemic Administration of NAC as an adjunct in the treatment of bioterror exposures such as anthrax, smallpox or radiation and for vaccination prophylaxis, and use in combination with DHEA” which is adopted and incorporated herein by reference. The Provisional Application 60/522,785 of Nov. 7, 2004 claimed the use of oral liposomal glutathione for the treatment of diseases such as cystic fibrosis and Parkinson's Disease and included demonstrations of the absorption of glutathione by laboratory and clinical observation.

In the in-vivo mammalian system, viral exposure to cells of the respiratory system results in responses from both the cells and the immune system cells defending the area. The lipoprotein structure of viruses contain structural components recognized by the immune system called antigens. The immune system contains a form of immunity called adaptive or acquired that refers to antigen-specific defense mechanisms that take several days to become protective and are designed to remove specific antigens. This is the immunity that one develops for life long protection. There are two major branches of the adaptive immune response: humoral and cell-mediated immunity.

Cell-mediated immunity involves the production of cytotoxic T-lymphocytes, activated macrophages, activated NK (Natural Killer) cells, and cytokines in response to an antigen and is mediated by T-lymphocytes. The cytokines released by the cells associated with this type of immune response are called Th-1.

Humeral immunity involves the production of antibody molecules in response to an antigen, and is mediated by B-lymphocytes. This type of response is characterized by cells that release cytokines associated with the Th-2 response.

Th-1-lymphocytes, the cellular immune response cells, recognize antigens such as viruses presented by macrophages and activate and increase cell-mediated immunity by producing cytokines such as interleukin-2 (IL-2), interferon-γ, (IFN-γ), lymphotoxin and tumor necrosis factor-α and β. These cytokines enable T8-lymphocytes to differentiate into cytotoxic T-lymphocytes capable of destroying infected host cells, as well as activating cytotoxic T-lymphocytes and NK cells.

The cytokines released by Th-2 lymphocytes include IL-2, 4, 5, 10, and 13 that promote antibody production. These cytokines enable and activate B-lymphocytes and result in the production of antigen specific antibodies.

The balance between these two subsets of lymphocytes plays a crucial role in how well the body defends against certain infections. For example, Th-1 cells are needed to produce IFN-γ, which prompts the release of TNF (Tumor Necrosis Factor). TNF encourages the formation of toxic forms of oxygen, called reactive oxygen species (ROS) that are capable of destroying microorganisms such as viruses. Conversely, the cytokines released by the Th-2 cells such as IL4 can actually slow the microbe killing activity related to IFN-γ.

Inflammatory states which persist for prolonged periods of time without resolving the triggering event and results in damage to cells and tissues are called chronic inflammation.

Infection with virus appears to create a state of oxidation stress in cells, even without the presence of immune cells. Evidence is accumulating that viral replication is dependent on a state of increased oxidation inside cells, where viral replication occurs. A shift toward a pro-oxidant state has been observed in the cells and body fluids of patients infected with human immunodeficiency virus (HIV), hepatitis C virus, as well as in the lungs of mice infected with the influenza virus.

During viral infection, the redox changes (increased oxidation) that occur have been demonstrated to be related to a depletion of glutathione, a depletion that varies in intensity, duration and mechanism depending on the type of virus and the host cell infected. Rapid decreases of glutathione have been demonstrated to occur with viruses that affect epithelial cells such as parainfluenza and Herpes simplex, and to parallel the progression of cell damage.

A deficiency of glutathione (reduced) may lead to damage to cells and tissues through several mechanisms including the accumulation of an excess of free radicals which causes disruption of molecules, especially lipids causing lipid peroxidation, and which combined with toxin accumulation will lead to cell death. These mechanisms are often referred to as oxidation stress as general term. The lack of sufficient glutathione in the reduced state relative to the oxidized state may be due to lack of production of glutathione (reduced) or an excess of the materials such as toxins that consume glutathione (reduced). The lack of glutathione (reduced) may manifest as a systemic deficiency or locally in specific cells undergoing oxidation stress.

Cytokines are a heterogeneous group of hormone-like proteins, produced by all organs and many cell types of the body that establish a communication network between various cells of each organ. In inflammatory diseases and ischemic processes, large amounts of cytokines are produced, causing edema, cellular metabolic stress, and finally tissue necrosis. The proinflammatory cytokines TNF-α, IL-1, IL-12, macrophage-inflammatory protein (MIP3)-1α, MIP-2, and IFN-γ are primarily involved in promoting inflammatory processes.

Most infections with respiratory viruses induce Th-1 responses characterized by the generation of Th-1 and CD8+ T cells secreting IFN-, which in turn have been shown to inhibit the development of Th-2 cells. Nasal lavage specimens from humans infected with influenza virus contain various proinflammatory cytokines, such as interleukin (IL)-6, TNF-α, gamma interferon (IFN-γ), IL-10, monocyte chemotactic protein 1, and macrophage inflammatory proteins 1α and 1β (1).

TNF-α expression in lung epithelial cells, which are the key targets of influenza virus infection, appears to be crucial to control of influenza virus infection in the host respiratory tract (1). The role this powerful inflammatory cytokine plays in recruiting various host cells, including monocytes and T and B lymphocytes, to sites of infection suggests that TNF-α plays an important role in clearing influenza virus infection in the respiratory tract before the secondary immune response is activated. Previous studies showed that influenza virus infection of human macrophages triggers production of TNF-α IL-1β, IL-18, and IFN-α/β. It is possible that CD4+ and CD8+ T cells can be very important sources of TNF-α, since these cells produce a considerable amount of TNF-α in an infected host. After infection, lung epithelial cells have been shown to produce TNF-α. Studies show that influenza virus infection of human macrophages triggers production of TNF-α, IL-1β, IL-18, and IFN-α/β. It is possible that CD4+ and CD8+ T cells can be very important sources of TNF-α, since these cells produce a considerable amount of TNF-α in an infected host.

At the same time that TNF-α affords a method of killing viruses or viral laden cells, the presence of TNF-α decreases the availability of glutathione.

TNF-α is produced by activated macrophages, T and B lymphocytes, natural killer cells, astrocytes, endothelial cells, smooth muscle cells, some tumor cells, and epithelial cells.

TNF-α factor is an inflammatory cytokine that causes damage by generation of oxidative stress. TNF-α has been shown to sensitize cells to injury from peroxide (H₂O₂). Peroxide is an oxidant produced by various cells responding to viral infection including polymorphonuclear cells, natural killer (NK) cells and T-killer cells. The presence of TNF-α even in low concentrations increases the permeability of cells, such as endothelial cells lining the respiratory tract, to damage from H₂O₂ peroxidation. The amount of reduced glutathione contained in cells has been shown to be decreased in a concentration-dependent fashion upon exposure to TNF-α.

It appears that TNF-α decreases the availability of reduced glutathione, resulting in an increase in local oxidation stress. The formation of the oxidized form of glutathione, GSSG, can accumulate when its rate of formation exceeds the cells ability to convert it back to reduced glutathione, GSH. In this situation, GSSG can be extruded out of the cell into the extracellular space, or can form mixed disulfides with intra or extracellular proteins resulting in a net loss of total glutathione inside the affected cell (2).

The resulting deficiency of glutathione leaves normal cells exposed to TNF-α induced peroxidation damage. Thus, the normal response of the immune system, in the presence of a glutathione deficiency, in fact exacerbates the symptomatic condition because the membrane of the normal cells becomes more susceptible to peroxidation damage. Peroxidation damage directed at diseased cells or infectious agents is a desired response; however, such damage directed at normal cells is undesirable.

When normal cells begin to suffer the peroxidation damage, the negative effects of TNF-α peroxidation and the reduction in cell glutathione can reinforce each other to the detriment of any cell. First, the release from the immune and epithelial cells of TNF-α is unregulated, and second, cells become progressively more sensitive to peroxidation damage as a result of continued TNF-α release, exacerbating local oxidative stress, often resulting in intensification of symptoms. In the most severe cases, the result is shock as seen in adult respiratory distress syndrome. In other cases, it may take several days for the body's normal combined response to viral infection to cause the symptoms to abate, which abatement the invention proposes to accelerate.

The normal response of a healthy cell is that glutathione will be upregulated. When that occurs, normal cells overcome the oxidative stress fairly easily. In many cases, however, either the local or systemic production of glutathione is insufficient to protect a normal cell under oxidative stress and the virus persists. In these situations, the invention enables more rapid resolution and amelioration of symptoms by providing normal supportive material for proper response by healthy cells.

A Th-1 response allows the NK and polymorphonuclear (PMN), e.g. macrophages, to consume virus in those cells so generalized release of TNF need not persist.

This invention is intended to use effective glutathione flow into cells to modulate the expected effects of TNF-alpha release.

Glutathione is required for the enzyme glutathione peroxidase, the enzyme that converts H₂O₂ to harmless molecules of water (H₂O). Thus, as the GSH level inside cells decreases, the susceptibility to H₂O₂ increases. Restoration of glutathione in cells in cell culture has been shown to increase the resistance of endothelial cells to H₂O₂. The present invention's ability to deliver glutathione to deficient immune cells as well as endothelial and epithelial cells is responsible for the improvement in symptoms observed by individuals with influenza as cited in the examples.

While it would appear that the situation of sudden loss of glutathione related to the release of TNF could be corrected by the addition of oral N-acetyl-cysteine (NAC) the rate limiting amino acid used in the formation of glutathione, there is a unique situation that occurs during inflammation that diminishes the efficacy of NAC in the formation of glutathione.

It has been demonstrated that inflammation related to the experimental injection of either lipopolysaccharide, an experimental tool used to mimic infection, or the injection of TNF will result in normal glutathione restoration in lung only if adequate dietary patterns were present prior to or during the infection (13). The dietary patterns included diets with adequate sulfur containing foods or the maintenance of a protein diet. During acute illnesses these dietary patterns may be absent.

The transsulferation pathway is used to form cysteine from homocysteine. The cysteine can then be used to form glutathione. There are several situations in which the pathway that normally forms glutathione from cysteine is shunted to the pathway that forms taurine, another sulfur bearing amino acid. It has been observed that this shunt to taurine occurs during acute inflammation or infection as seen in experimental sepsis in rats (17). Additionally there are other situations in which this shunt to taurine over glutathione occurs, including including HIV infection (15), and excess toxin exposure such as ethanol (16).

The findings in autism of low glutathione and elevated taurine excretion (unpublished observation) suggest that there are other situations in which the shunting of cysteine to glutathione exists as a significant contributing factor for disease. The anecdotally observed case of a 10 year old boy with chronic Epstein Barr Virus related disease, low glutathione and elevated taurine in the urine also point out that many inflammatory and infectious situations exist in which the use of NAC will not be the most efficient method of supporting the individual as the NAC will not necessarily be utilized in the formation of glutathione. Thus, the current invention becomes the preferred method for raising glutathione levels in individuals with infection and inflammation.

It is not possible with current technologies to measure the level of TNF-α, or GSH inside of cells of specific organs during infection with influenza. Thus, the monitoring of symptoms is the only method of observing responses to different remedies for the human system. The clinical improvement observed after the administration of the invention parallels the changes observed in in-vitro studies and provides powerful information about the probable mechanisms of both the symptoms of the illness and the effects of the invention.

TNF-α also has been demonstrated to play an important role in the pathogenesis of adult respiratory distress syndrome. This syndrome is associated with the development of pulmonary edema of non-cardiac origin and generally occurring in severely ill individuals. While lung damage due to damage to alveoli is the typical finding on tissue pathology examination, the diagnosis is usually made on clinical grounds as tissue for evaluation is rarely available during the illness. Increased edema in the alveoli results in decreased oxygenation. Recent research suggests a high association with TNF in the pathogenesis of ARDS. Other studies show that glutathione (reduced) is extremely low in the epithelial lining fluid of chronic lung diseases (Rahman (10)). This study showed that individuals with ARDS 31±8.4 mM of reduced glutathione compared to 651±103 mM in the controls and have an increase in oxidized glutathione. Oxidized glutathione is increased in the alveolar fluid of patients with the adult respiratory distress syndrome (ARDS) (Bunnell, 11). Replacing glutathione with either the intravenous form or the liposome encapsulation form will be of benefit in raising the level of reduced glutathione and ameliorating symptoms in ARDS.

The invention is also claimed as a method of treatment and prevention of ARDS.

Increased release of TNF has been implicated in a wide variety of inflammatory diseases including rheumatoid arthritis, Crohn's disease, multiple sclerosis, psoriasis, scleroderma, atopic dermatitis, systemic lupus erythematosus, type II diabetes, atherosclerosis, myocardial infarction, osteoporosis, and autoimmune deficiency disease.

Modulation of TNF is thus a desirable goal and medications called biologic response modifiers have been developed to try and block TNF activity. These medications include antibodies such as those used in the medications Remicade (infliximab) and Humira (Adalimumab) or soluble TNF receptors such as Enbrel (Etanercept) for use in diseases such as rheumatoid arthritis and Crohn's disease. However, since TNF is a critical component of effective immune surveillance and is required for proper function of NK cell, T cells, B cells, macrophages and dendritic cells, blocking TNF results in significant side effects. Such TNF blocking treatments increase the risk of serious, even fatal, infections, certain types of cancers and cardiotoxicity (18).

Thus, there is an urgent need for a biologic response modifier of TNF that are both safe and effective (18). The invention described, the liposomal encapsulation of glutathione is presented as a modulator of the response to TNF that is safe and effective.

Deficiency of glutathione in the cells which initially interact with invading material such as virus, which cells are called dendritic cells or antigen presenting cells, has been shown to influence the immune response state favoring the TH-2 response. In this situation there is a preponderance of response of the B-cell system with immune responses associated with the mediators of chronic inflammation.

The pro-oxidant state, whether previously established in a susceptible individual or created by the viral infection, contributes to the pathogenesis of virus-induced diseases by activating cytokines associated with the less efficient viral management state called chronic inflammation, characterized as TH-2 (4).

Restoring the level of antioxidant function in the immune regulating cells, such as macrophages and antigen presenting cells can be accomplished by increasing glutathione in these cells. Increased glutathione in these cells can return the overall immune response to a state of interaction of immune cells characterized by increasing the function of T cells, the TH-1 response (3).

Inflammation is associated with the generation of reactive oxygen intermediates (ROI), including superoxide anion (O2-), hydrogen peroxide (H2O2), and the hydroxyl radical (OH—). ROI, in addition to being efficient antimicrobial effector molecules, are also key mediators of inflammation (5). Providing adequate antioxidant protection by the present invention utilizing glutathione in liposomals may provide a rapid resolution of inflammation and the clinical symptoms associated with inflammation.

It is also possible that the present invention allows a more efficient immune function to occur affording the individual with a more rapid resolution of the viral infection, shortening the viral infection time interval. More efficient immune TH-1 function allows for more efficient management of viral infection and lessening or avoidance of flu-like symptoms.

It has also been demonstrated in cell cultures that viral infection triggers a series of steps starting with a decrease in available reduced glutathione, contributing to a decreased function of the Na+/H+ pump mechanism in the cell wall that maintains the level of H+ in the cell (14).

When the Na+/H+ pump becomes dysfunctional the level of H+ increases, resulting in an acidic state in the cell.

Increasing the level of ionized H+ results in a decrease in the pH of the cell. pH is a direct reflection in the number of H+ ions in the cell, represented as the reciprocal of the H+ concentration, a lower pH.

Illness states are associated with an increase in acidity (decreased pH), and it has been demonstrated that viral replication occurs more efficiently in cells that are acidic. Restoring the level of reduced glutathione may result in a restoration of the function of the cellular Na+/H+ pump, leading to a restoration of normal pH, resulting in rapid improvement in the symptoms the individual is experiencing.

### OBJECTS OF THE INVENTION

It is an object of the invention to modify immune function to create the situation in immune cells allowing a switch to a more efficient immune function such as the Th-1 response during viral infection, as well as situations with chronic inflammation.

It is an object of the invention to treat the deleterious effects of TNF alpha and beta in both acute and chronic conditions caused by virus as well as other infectious pathogens.

It is an object of the invention to be used in situations where general or immune cells have been primed by toxin exposure to release excessive TNF or the cells have developed an increased sensitivity to the effects of TNF or peroxides.

It is an object of the invention to utilize the liposomal encapsulation to deliver the reduced glutathione to the intracellular compartment of cells, and particularly macrophage, T-killer cells, and NK cells which are the cells which are the first line of response in defense against viral invaders like influenza.

It is an object of the invention to use intravenous infusion and maintain the glutathione in the reduced state, and to administer the glutathione in the liposomal formulation infused in isotonic or hypertonic concentrations to cause less irritation to veins and reduce the local vein irritation known as phlebitis.

### DETAILED DESCRIPTION OF THE PREFERRED EMBODIMENTS

### EXAMPLE 1

Liposomal glutathione Drink or Spray 2500 mg per ounce

| | % |
|---|---|
| | w/w |
| Deionized | 74.4 |
| Water | |
| Glycerin | 15.00 |
| Lecithin | 1.50 |
| Potassium | 0.10 |
| Sorbate | |
| (optional | |
| spoilage | |
| retardant) | |
| Glutathione | 8.25 |
| (reduced) | |

A lipid mixture having components lecithin, and glycerin were commingled in a large volume flask and set aside for compounding.

In a separate beaker, a water mixture having water, glycerin, glutathione were mixed and heated to 50.degree. C.

The water mixture was added to the lipid mixture while vigorously mixing with a high speed, high shear homogenizing mixer at 750-1500 rpm for 30 minutes.

The homogenizer was stopped and the solution was placed on a magnetic stirring plate, covered with parafilm and mixed with a magnetic stir bar until cooled to room temperature. Normally, a spoilage retardant such as potassium sorbate or BHT would be added. The solution would be placed in appropriate dispenser for ingestion as a liquid or administration as a spray.

Analysis of the preparation under an optical light microscope with polarized light at 400× magnification confirmed presence of both multilamellar lipid vesicles (MLV) and unilamellar lipid vesicles.

Notably, there is no requirement of cooling, or production at a certain temperature.

The preferred embodiment includes the variations of the amount of glutathione to create less concentrated amounts of glutathione. The methods of manufacture described in Keller et al, U.S. Pat. No. 5,891,465, Apr. 6, 1999, are incorporated into this description.

### EXAMPLE 2

Glutathione LipoCap Formulation

| Ingredient | Concentration (%) |
|---|---|
| Sorbitan Oleate | 2.0 |
| Glutathione | 89.8 |
| Purified Water | 4.0 |
| Potassium Sorbate | 0.2 |
| Polysorbate 20 | 2.0 |
| Phospholipon 90 (DPPC) | 2.0 |

Components are commingled and liposomes are made using the injection method (Lasic, D., Liposomes, Elsevier, 88-90, 1993). When liposome mixture cooled down 0.7 ml was drawn into a 1 ml insulin syringe and injected into the open-end of a soft gelatin capsule then sealed with tweezers. The resulting one gram capsule contains 898 IU of Vitamin E. Large scale manufacturing methods for filling gel caps, such as the rotary die process, are the preferred method for commercial applications.

General Dosing

The preferred dosing schedule of the invention for the treatment of influenza symptoms is 600 mg (land ½ teaspoon) of the invention to be taken at the first onset of symptoms. A dose of 400 mg (1 teaspoon) to 600 mg is to be repeated each hour until symptoms are relieved. Once symptom relief is achieved, the dose is repeated immediately upon the return of symptoms. The anticipated amount to be taken is 1 to 2 ounces in 24 hours. See case examples.

If symptoms recur in the following 24 hours the regimen may be repeated as stated.

1 ounce is 5.56 teaspoons.

1 teaspoon of the invention of oral liposomal glutathione reduced contains approximately 440 mg GSH.

A preferred mode sets a suggested dose based on body weight. Recommended amounts are for use in the treatment of influenza symptoms. For best results it is suggested that the invention be used at the early onset of flu symptoms of as a preventative after exposure the flu.

Gently stir liposomal glutathione into the liquid of your choice.

Determine Individual Dose by Body Weight: For Children

Under 30 lbs: ¼ teaspoon=100 mg GSH

30-60 lbs: ½ teaspoon=210 mg GSH

60-90 lbs: ¾ teaspoon=316 mg GSH

90-120 lbs: 1 teaspoon=422 mg GSH

120-150 lbs: 1½ teaspoon=630 mg GSH

Over 150 lbs: 1½ teaspoons=630 mg GSH

Dosing Schedule for the Treatment of Influenza Symptoms.

As stated, the initial dose should be according to body weight. For adults the dose is 1 and ½ teaspoon initially and repeat every 1 to 2 hours over 24 hour period.

The amount and frequency of doses may be decreased as the individual begins to improve. The period of treatment is usually 24 hours.

Ingestion of the liposomal preparation of reduced glutathione results in a rapid reduction in influenza symptoms as related in the examples cited. The mechanism may be related to one or more of the methods described. The rapid addition of reduced glutathione to the system by the invention has a number of avenues to facilitate restoration of normal general cell and immune cell function that results in the reduction of symptoms related to virus infection in general and including influenza.

Macrophage have a predilection to ingest particulate materials (6) such as liposomes, so the delivery of glutathione directly to these cells, responsible for directing immune responses, is particularly effective.

The invention has been demonstrated to have benefit in diseases associated with intracellular glutathione deficiency such as Parkinson's disease, and Cystic Fibrosis. These benefits have been claimed by this inventor in provisional application Ser. No. 60/522,785 on Nov. 7, 2004 entitled “Liposomal Formulation for Oral Administration of Glutathione (Reduced)” which is adopted and incorporated herein by reference, and in a presently pending application by F. T. Guilford, the inventor herein, U.S. Utility application Ser. No. 10/289,934 filed Nov. 7, 2002 entitled Systemic Administration of NAC as an adjunct in the treatment of bioterror exposures such as anthrax, smallpox or radiation and for vaccination prophylaxis, and use in combination with DHEA” which is adopted and incorporated herein by reference.

The clinically effective use of glutathione in its pure form directly without any additive encapsulation or transformation (in the “neat” form) has been previously limited to the intravenous administration of the biochemical in the reduced state. Demopoulus et al, U.S. Pat. No. 6,204,248, Mar. 20, 2001, describes the use of the glutathione in combination with crystalline ascorbic acid enclosed in a gel cap for oral administration to alter redox state of cells and improve disease processes. However, no suggestion is made of glutathione encapsulated in a liposome for the treatment of influenza, nor is any suggestion made as to the actual efficacy of glutathione delivery to the cell system set out.

A recent patent, Smith, U.S. Pat. No. 6,764,693, Jul. 20, 2004, claims the use of liposomes containing a combination of glutathione with at least one other antioxidant material to increase intracellular and extra cellular antioxidants. The Smith patent claims a mechanism of action of the liposome that involves the peroxidation and lysis of the liposome with resulting release of liposome content of the mixture of glutathione and other nutrients into the plasma.

By contrast, the preferred method of composition of the liposome claimed in this invention is for a liposome that functions by fusion or engulfing of the liposome into the cellular immune cell and transfer of the glutathione content into cells. Evidence for this method of action is provided in the clinical examples of improvement in the red blood cell level of glutathione paralleling clinical improvement in individuals with Cystic Fibrosis, F. T. Guilford provisional application Ser. No. 60/522,785 on Nov. 7, 2004 entitled “Liposomal Formulation for Oral Administration of Glutathione (Reduced)” which is referred to in the discussion earlier.

The preferred composition for oral use of the invention is for a liposome encapsulating only reduced glutathione, without other components.

Liposomes have been documented to fuse to cells such as red blood cells and deliver their content into the cells (7).

Another preferred mode is delivery of the liposomal glutathione is by placing the liposome containing glutathione into a gel cap. This allows a capsule delivery of unit dose. Capsule delivery facilitates storage, delivery and ingestion of the invention for many situations.

The liposome preparations claimed in this invention allow the manufacture of a stable product, which can be used for the administration of glutathione in a form that is convenient. The liposome-glutathione preparation described is also stable from oxidation. The preferred embodiment of the invention has been demonstrated to maintain glutathione in the reduced state, both after manufacture and at 14 months of storage at room temperature.

The preferred mode of the invention describes the lipid encapsulation of the glutathione (reduced) into the lipid vesicle of liposomes and administered orally for the transmucosal absorption into the nose, mouth, throat or gastrointestinal tract providing the ability to conveniently supply therapeutically effective amounts of glutathione (reduced). The invention may also be administered topically for dermal and transdermal administration, intravenously or in an encapsulation such as a gel cap.

Another form of the invention is the intravenous infusion of glutathione in solution for treatment in Adult Respiratory Distress Syndrome (ARDS) even if not in liposomal form. While Harbin et al, U.S. Pat. No. 6,835,811, Dec. 28, 2004, have reported a method of preparing glutathione in an intravenous solution, treatment for ARDS was not proposed. This invention provides for a considerably more stable liposomal formulation of glutathione than the less stable method in Harbin '811. The proposed uses in Harbin '811 for the less stabilized non-liposomal glutathione are uses of this invention of the stable formulation or solution of reduced glutathione in liposomes which can be used herein for oral administration and direct intravenous infusion.

The solution used for intravenous administration is prepared with glutathione concentrations of 200 mg per cc. The material is stored in vials of 10 cc for a total of 2000 mg per vial. The infusion may consist of 600 mg to 2000 mg given by rapid push infusion through an intravenous line. The infusion may be repeated on an hourly or as needed basis lessen the flu symptoms.

Providing the intravenous glutathione in a concentration that provides physiologic osmolarity is important. Osmolarity is a measure of the osmotic pressure exerted by a solution across a perfect semi-permeable membrane. Osmolarity is dependent on the number of particles in solution, but independent of the nature of the particles. The following table provides concentrations of glutathione in sterile water to create normal or hypertonic osmolarity. The average osmolarity of human serum is 290 mOsm. Solutions in the range of 240 to 340 mOsm are considered isotonic. Solutions that are hypotonic relative to cells have fewer dissolved solids or solutes than the interior of surrounding cells and results in fluid being pulled into cells. Thus, hypotonic fluids cause cells to swell and are considered dangerous to cells. Strategies for formulating concentrations of the fluids for intravenous infusion that create isotonic or hypertonic solutions are more desirable than using hypotonic solutions.

Examples utilizing “non-liposomal” “plain vanilla glutathione are as follows. The principles illustrated for resulting relative osmolarity are correlative to results using the composition of this invention.

RLG=Reduced L-Glutathione

| For Glutathione | | | |
|---|---|---|---|
| 2000 mg | Vol in ml | Milliosmoles/ml | Total Milliosmoles |
| RLG 200 mg/ml | 8.00 | 1.89 | 15.12 |
| Sterile water | 12.00 | 0.00 | 0.00 |
| Total | 20.00 | | 15.12 |
| Osmolarity: 856 | | | |

| For Glutathione | | | |
|---|---|---|---|
| 1000 mg | Vol in ml | Milliosmoles/ml | Total Milliosmoles |
| RLG 200 mg/ml | 5.00 | 1.89 | 9.45 |
| Sterile water | 20.00 | 0.00 | 0.00 |
| Total | 25.00 | | 9.45 |
| Osmolarity: 378 | | | |

| For Glutathione | | | |
|---|---|---|---|
| 600 mg | Vol in ml | Milliosmoles/ml | Total Milliosmoles |
| RLG 200 mg/ml | 3.00 | 1.89 | 5.67 |
| Sterile water | 15.00 | 0.00 | 0.00 |
| Total | 18.00 | | 5.67 |
| Osmolarity: 315 | | | |

No toxicity of glutathione has been reported. Amounts such as 1500 mg /m2 (Cancer (8) and 2500 mg daily for 5 days (9) have been reported to be well tolerated and reduce the effects of chemotherapy.

Case Examples and Dosing

Liposomal Glutathione in the Management of Influenza

Case 1. GG, a 59 year old woman in excellent general health developed symptoms of chills, low grade fever and ache all over consistent with the onset of influenza. The symptoms began at approximately 7 PM in the evening. The individual began the ingestion of liposomal glutathione with an initial dose of 600 mg (1 and ½ teaspoons). After one hour the symptoms were lessened, but still present and an additional 600 mg. of liposomal glutathione was ingested. This pattern continued for the next 4 hours with a total of 6 doses of land ½ teaspoons (600 mg) resulting in a total of 3600 mg. The individual noted significant reduction in clinical symptoms allowing a restful nights sleep.

In the morning GG noted some mild achiness and fatigue, but a significantly lessened set of symptoms. Liposomal glutathione was continued at a dose of 1 teaspoon (400 mg) every 2 hours for 3 additional doses. The individual noted at that time that the influenza symptoms were no longer present. The total amount ingested was approximately 2 ounces or 5000 mg. in the 18 hour period until the resolution of the symptoms.

Case 2.

Chris T is a 37 year old man who presents with fatigue, weakness, diaphoresis, pallor and a sense of exhaustion. The symptoms had been present and progressing over a 14 day period of time, following an episode described as a “bad flu”. At the time of evaluation at 10 AM he was considering returning to bed as even light lifting tasks and standing as part of his sales job was exhausting.

600 mg of oral liposomal glutathione was administered and the individual observed. He noted that approximately 45 minutes after ingesting the invention his symptoms began to lessen. His color returned to normal, the diaphoresis ceased and he felt a significant return of energy and strength. The improvement lasted almost an hour when his symptoms began to return.

Chris T. repeated the 600 mg dose and 20 to 30 minutes later again felt resolution of his symptoms. He repeated this schedule every 1 to 2 hours through the day. By 8 PM he had ingested 1 and ½ ounces (approximately 3750 mg) of the invention and his symptoms had resolved completely. Using the invention through the day, he was able to complete his sales job, which on that day included standing all day, some light lifting of his product and interacting with customers continually through the day.

The next morning in this example 2, Chris T., reported that his flu symptoms had abated. However, his sales companion, S? was now reporting the onset of similar flu symptoms.

Example 3, S., a 39 year old woman, developed the onset of symptoms including head and body ache, fatigue, low grade fever, mild pallor, and mild diaphoresis approximately 2 hours prior to evaluation. 600 mg of the invention was ingested orally. Approximately 40 minutes later she reported significant improvement in her symptoms. She reports that she continued the regimen of repeating ingestion of 400 to 600 mg of the invention every 1 to 2 hours. She ingested approximately 3750 mg that day and continued the protocol with doses of 400 mg every two to three hours the next day. The flu symptoms did not progress, and while she felt mild symptoms for another day or two, the symptoms of influenza never progressed beyond the symptoms she experienced at the onset.

The embodiments represented herein are only a few of the many embodiments and modifications that a practitioner reasonably skilled in the art could make or use. The invention is not limited to these embodiments. Alternative embodiments and modifications which would still be encompassed by the invention may be made by those skilled in the art, particularly in light of the foregoing teachings. Therefore, the following claims are intended to cover any alternative embodiments, modifications or equivalents which may be included within the spirit and scope of the invention as claimed.

### REFERENCES

1. Seo S H, Webster R G. Tumor necrosis factor alpha exerts powerful anti-influenza virus effects in lung epithelial cells. Journal of Virology February 2002; 76(3): 1071-1076. PMID: 11773383

2. Ishii Y, Partridge C A, Del Vecchio P J, Malik A B. Tumor necrosis factor-alpha-mediated decrease in glutathione increases the sensitivity of pulmonary vascular endothelial cells to H2O2. Journal Clinical Investigation March 1992;89(3):794-802. PMID: 1541673

3. Peterson J D, Herzenberg L A, Vasquez K, Waltenbaugh C. Glutathione levels in antigen-presenting cells modulate Th-1 versus Th-2 response patterns. Proceedings National Academy Science USA. Mar. 17, 1998; 95(6): 3071-3076. PMID: 9501217

4. Droge W, Schulze-Osthoff K, Mihm S, et al. Functions of glutathione and glutathione disulfide in immunology and immunopathology. Federation of American Scientists in Experimental Biology Journal 1994; Volume 8:1 pages 131-1138. PMID 7958618.

5. Saccani A, Saccani S, Orlando S, Sironi M, Bernasconi S, Ghezzi P, Mantovani A, Sica A. Redox regulation of chemokine receptor expression. Proceedings National Academy Science USA. Mar. 14, 2000; 97(6): 2761-2766. Immunology. PMID: 10716998.

6. Van Rooijen N, The liposome-mediated macrophage ‘suicide’ technique. Journal Immunological Methods. Nov. 13, 1989;124(1):1-6. PMID: 2530286

7. Constantinescu I, Liposomes and blood cells: a flow cytometric study. Artificial cells, blood substitutes, and immobilization biotechnology, November 2003;31(4):395-424. PMID: 14672416

8. Di Re F, Bohm S, Oriana S, Spatti G B, Zunino F Efficacy and safety of high-dose cisplatin and cyclophosphamide with glutathione protection in the treatment of bulky advanced epithelial ovarian cancer Cancer Chemotherapy and Pharmacology 1990;25(5):355-60. PMID 2306797

9. Bohm S, Oriana S, Spatti G, Di Re F, Breasciani G, Pirovano C, Grosso I, Martini C, Caraceni A, Pilotti S, Zunino F Dose intensification of platinum compounds with glutathione protection as induction chemotherapy for advanced ovarian carcinoma. Oncology. 1999;57(2):115-20. PMID: 10461057

10. Rahman I, MacNee W. Oxidative stress and regulation of glutathione in lung inflammation. Eur Respir J. September 2000;16(3):534-54. PMID: 11028671

11. Bunnel E, Pacht E R, American Review Respiratory Disease 1993; Vol. 148: pgs. 1174-1178). PMID 8239150

12. Ciriolo M R, Palamara A T, Incerpi S, Lafavia E, Bue M C, De Vito P, Garaci E, Rotilio G. Loss of GSH, oxidative stress, and decrease of intracellular pH as sequential steps in viral infection. J Biol Chem. Jan. 31, 1997;272(5):2700-8.

13. Hunter E A, Grimble R F. Dietary sulphur amino acid adequacy influences glutathione synthesis and glutathione-dependent enzymes during the inflammatory response to endotoxin and tumour necrosis factor-alpha in rats. Clinical Science (London). March 1997;92(3):297-305. PMID: 9093011

14. Cutaia M, Parks N. Oxidant stress decreases Na+/H+ antiport activity in bovine pulmonary artery endothelial cells. Am J Physiol. December 1994;267(6 Pt 1):L649-59.

15. Hortin G L, Landt M, Powderly W G. Changes in plasma amino acid concentrations in response to HIV-1 infection. Clinical Chemistry. May 1994;40(5):785-9. PMID: 7909726

16. Jung Y S, Kwak H E, Choi K H, Kim Y C. Effect of acute ethanol administration on S-amino acid metabolism: increased utilization of cysteine for synthesis of taurine rather than glutathione. Advances in Experimental Medicine and Biology. 2003;526:245-52. PMID: 12908607

17. Santangelo F. The regulation of sulphurated amino acid junctions: fact or fiction in the field of inflammation? Amino Acids. 2002;23(4):359-65. Review. PMID: 12436203

18. Aggarwal B B, Shishodia S, Takada Y, Jackson-Bernitsas D, Ahn K S, Sethi G, Ichikawa H. TNF blockade: an inflammatory issue. Ernst Schering Research Foundation Workshop. 2006;(56):161-86. Review. PMID: 16331857

What is claimed is:

1. A pharmaceutical composition enabling delivery after oral or intravenous administration of a therapeutically effective amount of glutathione (reduced) to a mammalian patient comprising:
a therapeutic dose of reduced glutathione stabilized in a liposomal pharmaceutical carrier capable of being ingested orally, and notwithstanding that oral administration, capable of delivering glutathione (reduced) in a physiologically active state to improve symptoms of disease.

2. The composition according to claim 1, further comprising:
a pharmaceutically acceptable form of Selenium.

3. The composition according to claim 1, further comprising:
said liposomal pharmaceutical carrier having a gel selected from the group of non-oxidizing edible gels, including glycerin.

4. The composition according to claim 1, further comprising:
a pharmaceutically acceptable form of Selenium;
and said liposomal pharmaceutical carrier having a gel selected from the group of non-oxidizing edible gels, including glycerin.

5. The composition according to claim 1, further comprising:
said disease state being characterized by either acute or chronic inflammation characterized by release of Tumor Necrosis Factor-alpha (TNF-α).

6. The composition according to claim 5, further comprising:
a pharmaceutically acceptable form of Selenium.

7. The composition according to claim 5, further comprising:
said liposomal pharmaceutical carrier having a gel selected from the group of non-oxidizing edible gels, including glycerin.

8. The composition according to claim 5, further comprising:
a pharmaceutically acceptable form of Selenium;
and said liposomal pharmaceutical carrier having a gel selected from the group of non-oxidizing edible gels, including glycerin.

9. A pharmaceutical composition enabling delivery after oral or intravenous administration of a therapeutically effective amount of glutathione (reduced) to a mammalian patient comprising:
a therapeutic dose of reduced glutathione stabilized in a liposomal pharmaceutical carrier capable of being ingested orally, and notwithstanding that oral administration, capable of delivering glutathione (reduced) in a physiologically active state to improve symptoms in disease states characterized by either acute or chronic inflammation by transfer of the glutathione into cells of said patient.

10. The composition according to claim 9, further comprising:
a pharmaceutically acceptable form of Selenium.

11. The composition according to claim 9, further comprising:
said liposomal pharmaceutical carrier having a gel selected from the group of non-oxidizing edible gels, including glycerin.

12. The composition according to claim 9, further comprising:
a pharmaceutically acceptable form of Selenium;
and said liposomal pharmaceutical carrier having a gel selected from the group of non-oxidizing edible gels, including glycerin.

13. The composition according to claim 9, further comprising:
said disease state being acute or chronic inflammation being characterized by release of Tumor Necrosis Factor-alpha (TNF-α).

14. The composition according to claim 13, further comprising:
a pharmaceutically acceptable form of Selenium.

15. The composition according to claim 13, further comprising:
said liposomal pharmaceutical carrier having a gel selected from the group of non-oxidizing edible gels, including glycerin.

16. The composition according to claim 13, further comprising:
a pharmaceutically acceptable form of Selenium;
and said liposomal pharmaceutical carrier having a gel selected from the group of non-oxidizing edible gels, including glycerin.

17. The composition according to claim 9, further comprising:
said disease state being viral-related illness.

18. The composition according to claim 17, further comprising:
a pharmaceutically acceptable form of Selenium.

19. The composition according to claim 17, further comprising:
said liposomal pharmaceutical carrier having a gel selected from the group of non-oxidizing edible gels, including glycerin.

20. The composition according to claim 17, further comprising:
a pharmaceutically acceptable form of Selenium;
and said liposomal pharmaceutical carrier having a gel selected from the group of non-oxidizing edible gels, including glycerin.

21. The composition according to claim 9, further comprising:
said disease state being an upper respiratory tract infection.

22. The composition according to claim 21, further comprising:
a pharmaceutically acceptable form of Selenium.

23. The composition according to claim 21, further comprising:
said liposomal pharmaceutical carrier having a gel selected from the group of non-oxidizing edible gels, including glycerin.

24. The composition according to claim 21, further comprising:
a pharmaceutically acceptable form of Selenium;
and said liposomal pharmaceutical carrier having a gel selected from the group of non-oxidizing edible gels, including glycerin.

25. The composition according to claim 9, further comprising:
said disease state being Acute Respiratory Distress syndrome (ARDS).

26. The composition according to claim 25, further comprising:
a pharmaceutically acceptable form of Selenium.

27. The composition according to claim 25, further comprising:
said liposomal pharmaceutical carrier having a gel selected from the group of non-oxidizing edible gels, including glycerin.

28. The composition according to claim 25, further comprising:
a pharmaceutically acceptable form of Selenium;
and said liposomal pharmaceutical carrier having a gel selected from the group of non-oxidizing edible gels, including glycerin.

29. A pharmaceutical composition enabling delivery after oral or intravenous administration of a therapeutically effective amount of glutathione (reduced) to a mammalian patient with Acute Respiratory Distress syndrome (ARDS) comprising:
a therapeutic dose of reduced glutathione stabilized in a liposomal pharmaceutical carrier capable of being ingested orally, and notwithstanding that oral administration, capable of delivering glutathione (reduced) in a physiologically active state to improve symptoms associated with Acute Respiratory Distress syndrome (ARDS) by transfer of the glutathione into cells of said patient.

30. The composition according to claim 29, further comprising:
a pharmaceutically acceptable form of Selenium.

31. The composition according to claim 29, further comprising:
said liposomal pharmaceutical carrier having a gel selected from the group of non-oxidizing edible gels, including glycerin.

32. The composition according to claim 29, further comprising:
a pharmaceutically acceptable form of Selenium;
and said liposomal pharmaceutical carrier having a gel selected from the group of non-oxidizing edible gels, including glycerin.

33. The composition according to claims 1 through 32, further comprising:
said therapeutic dose being augmented by repeated administration of said composition stabilized in a liposomal pharmaceutical carrier capable of being ingested orally, and repeated as required until said animal reaches a stable state of health.

34. The composition according to claims 1 through 32, further comprising:
an anti-flu drug.

35. The composition according to claims 1 through 32, further comprising:
an anti-flu drug selected from the group of influenza vaccines and amantadine, rimantadine, and oseltamivir.

36. A method of treatment of a mammalian patient having a disease state
characterized by release of Tumor Necrosis Factor-alpha (TNF-α), comprising:
administering to said mammalian patient a therapeutic dose of reduced glutathione stabilized in a liposomal pharmaceutical carrier capable of being ingested orally or administered intravenously or parenterally, and notwithstanding that oral administration, capable of delivering glutathione (reduced) in a physiologically active state by transfer of the glutathione into cells of said patient.

37. The method of treatment according to claim 36, further comprising:
a pharmaceutically acceptable form of Selenium.

38. The method of treatment according to claim 36, further comprising:
said liposomal pharmaceutical carrier having a gel selected from the group of non-oxidizing edible gels, including glycerin.

39. The method of treatment according to claim 36, further comprising:
a pharmaceutically acceptable form of Selenium;
and said liposomal pharmaceutical carrier having a gel selected from the group of non-oxidizing edible gels, including glycerin.

40. A method of treatment of a mammalian patient having characterized by either acute or chronic inflammation, comprising:
administering to said mammalian patient a therapeutic dose of reduced glutathione stabilized in a liposomal pharmaceutical carrier capable of being ingested orally or administered intravenously or parenterally, and notwithstanding that oral administration, capable of delivering glutathione (reduced) in a physiologically active state by transfer of the glutathione into cells of said patient.

41. The method of treatment according to claim 40, further comprising:
said therapeutic dose having a pharmaceutically acceptable form of Selenium.

42. The method of treatment according to claim 40, further comprising:
said liposomal pharmaceutical carrier having a gel selected from the group of non-oxidizing edible gels, including glycerin.

43. The method of treatment according to claim 40, further comprising:
said therapeutic dose having a pharmaceutically acceptable form of Selenium;
and said liposomal pharmaceutical carrier having a gel selected from the group of non-oxidizing edible gels, including glycerin.

44. A method of treatment of a mammalian patient having viral-related illness, comprising:
administering to said mammalian patient a therapeutic dose of reduced glutathione stabilized in a liposomal pharmaceutical carrier capable of being ingested orally or administered intravenously or parenterally, and notwithstanding that oral administration, capable of delivering glutathione (reduced) in a physiologically active state to cells of said patient to improve symptoms of viral disease.

45. The method of treatment according to claim 44, further comprising:
said therapeutic dose having a pharmaceutically acceptable form of Selenium.

46. The method of treatment according to claim 44, further comprising:
said liposomal pharmaceutical carrier having a gel selected from the group of non-oxidizing edible gels, including glycerin.

47. The method of treatment according to claim 44, further comprising:
said therapeutic dose having a pharmaceutically acceptable form of Selenium;
and said liposomal pharmaceutical carrier having a gel selected from the group of non-oxidizing edible gels, including glycerin.

48. A method of treatment of a mammalian patient having adult respiratory distress syndrome (ARDS), particularly a geriatric patient, comprising:
administering to said mammalian patient a therapeutic dose of reduced glutathione having adult respiratory distress syndrome (ARDS) intravenously or parenterally in a physiologically active state.

49. The method of treatment according to claim 48, further comprising:
said therapeutic dose having a pharmaceutically acceptable form of Selenium.

50. The method of treatment according to claim 48, further comprising:
said liposomal pharmaceutical carrier having a gel selected from the group of non-oxidizing edible gels, including glycerin.

51. The method of treatment according to claim 48, further comprising:
said therapeutic dose having a pharmaceutically acceptable form of Selenium;
and said liposomal pharmaceutical carrier having a gel selected from the group of non-oxidizing edible gels, including glycerin.

52. A method of treatment of a mammalian patient having adult respiratory distress syndrome (ARDS), particularly a geriatric patient, comprising:
administering to said mammalian patient a therapeutic dose of reduced glutathione stabilized in a liposomal pharmaceutical carrier capable of being ingested orally or administered intravenously or parenterally, and notwithstanding that oral administration, capable of delivering glutathione (reduced) in a physiologically active state by transfer of the glutathione into cells of said patient.

53. The method of treatment according to claim 52, further comprising:
said therapeutic dose having a pharmaceutically acceptable form of Selenium.

54. The method of treatment according to claim 52, further comprising:
said liposomal pharmaceutical carrier having a gel selected from the group of non-oxidizing edible gels, including glycerin.

55. The method of treatment according to claim 52, further comprising:
said therapeutic dose having a pharmaceutically acceptable form of Selenium;
and said liposomal pharmaceutical carrier having a gel selected from the group of non-oxidizing edible gels, including glycerin.

56. The method of treatment according to claims 36 through 55, further comprising:
repeatedly administering said therapeutic dose as required until said animal reaches a stable state of health.

57. The method of treatment according to claims 36 through 55, further comprising:
said therapeutic dose having an anti-flu drug.

58. The method of treatment according to claims 36 through 55, further comprising:
said therapeutic dose having an anti-flu drug selected from the group of influenza vaccines and amantadine, rimantadine, and oseltamivir.

59. The use of a pharmaceutical composition according to claims 1-35.
